(19) United States
(12) Patent Application Publication
(10) Pub. No.: US 2021/0093687 A1
(43) Pub. Date: Apr. 1, 2021

(54) Composition for the Modulation of Circadian Rhythmicity and Sleep Quality

(71) Applicant: Savant Science Inc., Coral Springs, FL (US)

(72) Inventor: Jennifer P Cooper, Lindon, UT (US)

(21) Appl. No.: 17/039,426

(22) Filed: Sep. 30, 2020

Related U.S. Application Data
(60) Provisional application No. 62/908,231, filed on Sep. 30, 2019.

(51) Int. Cl. A61K 36/8965; A61K 36/75; A61K 31/366; A61K 36/185; A61K 36/81; A61K 36/9066; A61K 36/53; A61K 31/198; A61K 31/197; A61K 36/575; A61K 36/756; A61K 31/405; A61K 36/48; A61K 36/42; A61K 31/675; A61K 33/06; A61K 31/4045; A61K 36/24; A61K 36/65; A61K 31/216; A61K 36/537; A61K 31/4172

(52) U.S. Cl. CPC A61K 36/8965; A61K 31/4172; A61K 31/366; A61K 36/185; A61K 36/81; A61K 36/9066; A61K 36/53; A61K 31/198; A61K 31/197; A61K 36/575; A61K 36/756; A61K 31/405; A61K 36/48; A61K 36/42; A61K 31/675; A61K 33/06; A61K 31/4045; A61K 36/24; A61K 36/65; A61K 31/216; A61K 36/537; A61K 36/75

(57) ABSTRACT

A composition of matter in the form of a base formula that is specifically formulated to be effective to overcome sleep issues. Optional additions to the base formula are disclosed to be effective in the improvement of specific sleep issues.

### CROSS-REFERENCE TO RELATED APPLICATIONS

This application is a non-provisional of, and claims priority to, U.S. Application Ser. No. 62/908,231, titled “Composition for the Modulation of Circadian Rhythmicity and Sleep Quality,” filed on Sep. 30, 2019; which application is incorporated by reference herein in its entirety.

### FIELD OF THE INVENTION

The present invention generally relates to sleep products, and more specifically to a composition of matter specially formulated as a sleep aid to restore a circadian rhythm.

### BACKGROUND OF THE INVENTION

Circadian rhythms regulate many biological processes and play fundamental roles in behavior, physiology, and metabolism. Such periodicity is critical for homeostasis because disruption or misalignment of the intrinsic rhythms is associated with the onset and progression of various human diseases and often directly leads to pathological states. Since the first identification of mammalian circadian clock genes, numerous genetic and biochemical studies have revealed the molecular basis of these cell-autonomous and self-sustainable rhythms.

Circadian rhythms are ubiquitous biological oscillations with an approximate 24-hour period. These evolutionarily well-conserved rhythms arise from an intrinsic timekeeping system known as the “circadian clock,” which allows organisms to anticipate environmental cycling and coordinate biological processes. This clock is self-sustainable through an elaborate cooperation of genetic components and is hierarchically organized into a circadian timing system. In mammals, the apex of this system is the suprachiasmatic nucleus (SCN) of the hypothalamus, which is considered the central or master clock. The SCN integrates environmental cues such as light into time information to entrain its phase and then conveys this information to other oscillators in extraSCN brain regions and peripheral tissues. Indeed, in multicellular organisms, most cells harbor cell-autonomous oscillators.

These so-called local or peripheral clocks contribute to overt circadian rhythms, including the rest-activity cycle, periodic daily variations in metabolism and body temperature, as well as rhythmic hormone secretion. Robust circadian timing is required for health, and disruption of these intrinsic rhythms causes diverse pathologies. For instance, circadian disruption caused by shift-work, jet-lag, social “jet-lag”, digital “jet lag,” mis-timed food intake, stress, illness, and other variability or variable within the wake/sleep cycle is considered a risk factor for various chronic diseases, including sleep disorders, metabolic syndromes, cardiovascular diseases, affective disorders, neurodegeneration, and tumorigenesis. Much work is done on the pharmacological side to develop therapeutics related to modulating circadian rhythm for specific disease applications. However, there is further need to develop nutritional support to synchronize and promote regularity within the circadian clock as part of a healthy lifestyle and for overall health.

This misalignment of central and peripheral clocks may be involved in the development of disease and the acceleration of aging, possibly in a gender-specific manner. Recent evidence strongly points to the ubiquitous influence of circadian timing in nearly all physiologic functions. Thus, in addition to the prominent sleep and wake disturbances, circadian rhythm disorders are associated with cognitive impairment, mood disturbances, and increased risk of cardiometabolic disorders.

Two primary biological systems control sleep: the circadian timing system and homeostatic control. It was previously believed that sleep architecture was exclusively under homeostatic control. However, recent research has shown that the circadian timing system plays an important role in both SWs and REM sleep.

Slow brain waves (SWs, 0.5-4 Hz) are characteristic of a key component of the normal sleep cycle. SWs decline during sleep and this is thought to be related to a reduction of synaptic strength in cortical networks and to be central to sleep's role in maintaining brain function. A central assumption in current concepts of sleep function is that during sleep, SWs, and associated recovery processes, are independent of circadian rhythmicity. However, new data shows that circadian rhythmicity significantly modulated the incidence, amplitude, frequency and the slope of the SWs such that the peaks of the circadian rhythms in these slow-wave parameters were located during the biological day. Circadian rhythmicity directly modulates characteristics of SWs thought to be related to synaptic plasticity.

Additionally, the daily timing of rapid eye movement (REM) sleep reflects an interaction between the circadian pacemaker located in the suprachiasmatic nucleus of the hypothalamus (SCN) and a homeostatic process that induces compensatory REM sleep in response to REM sleep loss. Whether the circadian variation in REM sleep propensity is caused by active promotion, inhibition, or passive gating of REM sleep homeostasis by the SCN is unknown. Studies show that transitions into REM sleep are facilitated by the SCN during the rest phase, but the amount of REM sleep, once initiated, is determined primarily by homeostatic mechanisms. This work supports a role for the SCN in the active promotion of REM sleep at specific times of day.

Actigraphy is a procedure that records and integrates the occurrence and degree of limb movement activity over time. Actigraphic devices can be worn on the wrist, ankle or waist, relatively unobtrusively over a period of days to weeks. For sleep applications, the devices are typically worn on the wrist or ankle. Mathematical algorithms are then applied to these data to estimate wakefulness and sleep. In addition to providing a graphical summary of wakefulness and sleep patterns over time (i.e., temporal raster plots), actigraphy generates estimates of certain sleep parameters that are also commonly estimated by using sleep logs, or measured directly by polysomnography (PSG), the gold standard measure of sleep.

The sleep parameters estimated by actigraphy, in common with standard sleep logs, include: sleep latency (SL); total sleep time (TST); wake after sleep onset (WASO); and sleep efficiency (SE; SE=TST/time in bed). Actigraphy provides consistent objective data that is often unique from patient-reported sleep logs for some sleep parameters in adult and pediatric patients with suspected or diagnosed insomnia, circadian rhythm sleep-wake disorders, sleep-disordered breathing, central disorders of hypersomnolence, and adults with insufficient sleep syndrome.

### SUMMARY OF THE INVENTION

Briefly, according to a preferred embodiment, we disclose a base formula that is a composition of matter in the form of a circadian rhythm sleep aid specifically formulated to be effective to overcome sleep issues. Sleep quality is defined as satisfaction from sleep in which dimensions such as beginning of sleep, maintaining sleep, sleep quantity and how refreshed one is when waking.

Unlike current sleep aids that simply induce a state of unconsciousness, the disclosed base formula composition is specially formulated to correct any imbalances in a user's circadian rhythm to promote a healthy sleep cycle. Embodiments of the invention target specific sleep dysfunctions to allow the body to get restorative sleep. Each embodiment is uniquely formulated by adding to the common base formula, or composition, in order to be effective for achieving different sleep needs.

Scientific studies conducted by the Applicant have shown unexpected benefits to the user that include, but are not limited to:

1. Address unique sleep concerns using individual, customized formulas based upon a common base composition, that target individual needs;

2. A specially formulated composition of natural ingredients provides nutrients that the body needs to achieve regenerative and restorative sleep;

3. Gets the natural circadian rhythm back in sync;

4. Improved sleep quality, including: fall asleep faster; wake less at night; have less disruptive waking at night; feel more rested in the morning; have more energy during the day; feel ready for bed at night (tiredness at appropriate times);

5. Improve circadian rhythmicity;

6. Restore period regularity within the circadian rhythm;

7. Support circadian synchronization;

8. Reinforce circadian amplitude of key circadian variables;

9. Promote or deepen Slow Wave Sleep; and

10. Conserve REM sleep.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a simplified block diagram representing the various blends that are based upon the base formula composition;

FIG. 2 is a table of Circadian Timing Blend ingredients and their dosage ranges, according to an embodiment; and

FIG. 3 shows an exemplary product label listing the ingredients in the “fall asleep” blend, according to an embodiment.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

We discuss a composition of matter in the form of a circadian rhythm sleep aid specifically formulated to be effective to overcome sleep issues. Additional embodiments of the composition introduce the addition of optional ingredients chosen to target specific sleep dysfunctions to allow the body to get restorative sleep.

The Applicant has conducted a sleep study to determine the efficacy of the invention. The study measured sleep quality and circadian rhythmicity and sleep quality by completing clinically validated and recognized standard questionnaires for evaluating circadian variables. In the study, half of the study's subjects described their sleep problem as lack of feeling rested upon waking. Furthermore, 25% of subjects reported that they had problems falling asleep. 25% of subjects reported their primary sleep concern was repeated nighttime waking. This study showed unexpected results in improving slow wave sleep, not as a function of merely increasing sleep time (which we know is flawed from non-benzodiazepine sleep aids) or stealing it from REM sleep as is commonly seen when making up a sleep deficit, but from managing it via mediating multiple circadian rhythm attributes through the use of the disclosed composition.

The disclosed composition supports the circadian timing system in human beings as measured by sleep parameters, including sleep duration, sleep efficiency, sleep waking and sleep latency.

### Base Formula

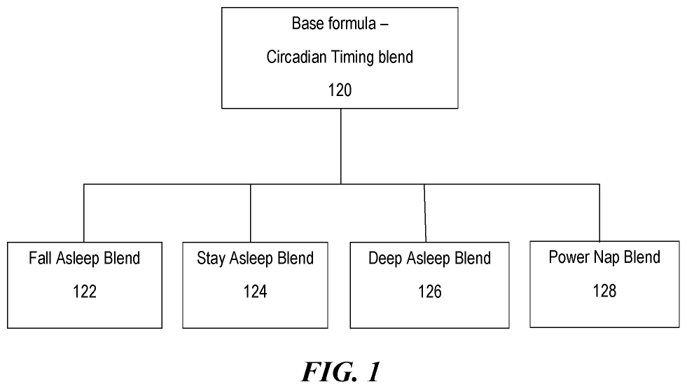

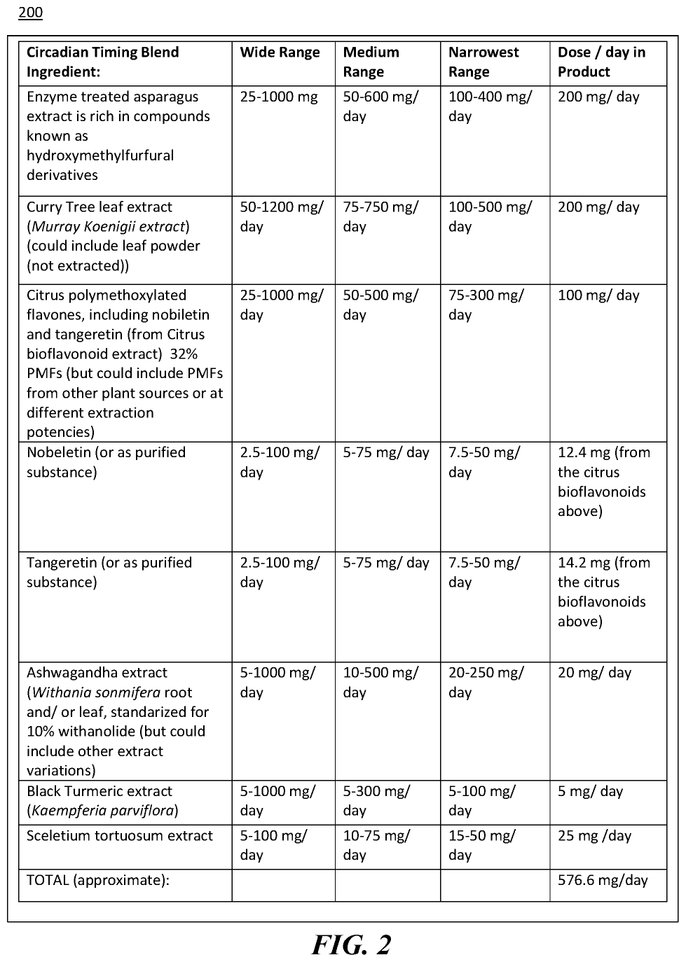

Referring now to FIG. 1, we disclose an embodiment of the invention, a base formula, Circadian Timing Blend 120. The Circadian Timing Blend 120 consists essentially of the following main ingredients preferably contained in one serving (two capsules): Enzyme treated asparagus extract, curry tree leaf extract (Murray Koenigii extract), Citrus polymethoxylated flavones, including nobiletin and tangeretin (from Citrus bioflavonoid extract), Sceletium tortuosum extract, Ashwagandha extract (Withania somnifera root and leaf (standardized to 10% withanolide), black turmeric extract (Kaempferia parviflora). The suggested dosage amounts for each ingredient of the Circadian Timing Blend 120 are provided in Table 1 200 of FIG. 2.

### Targeted Formulas

Depending on the targeted sleep issue, other embodiments combine additional ingredients to the Circadian Timing Blend 120 to formulate other compositions effective to address specific sleep concerns. These additional ingredients can account for 50%-70% of the total composition ingredients.

For example, a “FALL ASLEEP” blend 122 adds Melissa officinalis (L) from lemon balm extract, L-Theanine, and Gamma-aminobutyric acid (GABA). Someone who finds it difficult to fall asleep will benefit from taking two capsules 30 to 45 minutes before bedtime.

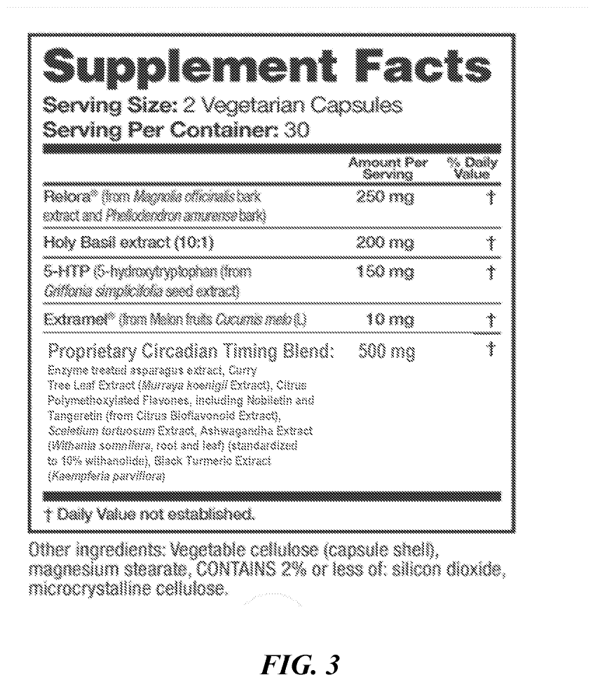

A “STAY ASLEEP” blend 124 adds Magnolia officinalis bark extract and Phellodendron amurense bark, Holy Basil extract, 5-hydroxytryptophan (5-HTP) from Griffonia simplicifolia seed extract, and Cucumis melo (L). FIG. 3 shows an example of the product label listing the ingredients and amounts per serving. This blend 124 provides the nutrients you need to help you stay asleep by supporting the transition from slow-wave (light) sleep to REM sleep.

A “DEEP ASLEEP” blend 126 adds Vitamin B6, Magnesium, Melatonin, GABA, Apocynum venetum leaf extract, and White Peony Extract (Paeonia lactiflora). Deep sleep is vital to recover from daily wear and tear on the brain and body. This formulation allows you to successfully enter the deep sleep cycle.

A “POWER NAP” blend 128 adds Melissa officinalis (L) leaves, 6% rosmarinic acid, Salvia lavandulifolia and Salvia officinalis, L-Theanine, L-ergothioniene. Power naps as short as 20 minutes have been shown to improve energy, increase mental focus and creativity, improve memory, and enhance physical performance. The “POWER NAP” blend 128 is a non-drowsy formula that is most effective when taken in conjunction with a 20-minute nap.

Although the present invention may be utilized in many different treatment modalities, it is discussed herein as an over-the-counter (OTC) sleep aid medication for compactness of disclosure. Without limiting the invention, a preferred embodiment of the invention is a medicine to be taken orally, such as a tablet, capsule, chewable tablet, or lozenge. Other forms of ingestion are contemplated within the spirit and scope of the disclosure. One with knowledge in the art will appreciate that the above-described formulas can be combined with inactive ingredients as required for chemical stability and to enhance flavor, appearance, and digestibility. It should be understood that one formula, or embodiment, can be combined with another embodiment.

What is claimed is:

1. A circadian rhythm sleep aid effective to overcome sleep issues consisting essentially of:
enzyme treated asparagus extract;
curry tree leaf extract;
citrus polymethoxylated flavones;
sceletium tortuosm extract;
ashwagandha extract; and
black turmeric extract.

2. The circadian rhythm sleep aid of claim 1, adding Melissa officinalis (L) from lemon balm extract, L-Theanine, and Gamma-aminobutyric acid (GABA).

3. The circadian rhythm sleep aid of claim 1, adding Magnolia officinalis bark extract, Phellodendron amurense bark, Holy Basil extract, 5-hydroxytryptophan (5-HTP) from Griffonia simplicifolia seed extract, and Cucumis melo (L).

4. The circadian rhythm sleep aid of claim 1, adding Vitamin B6, Magnesium, Melatonin, GABA, Apocynum venetum leaf extract, and White Peony Extract (Paeonia lactiflora).

5. The circadian rhythm sleep aid of claim 1, adding Melissa officinalis (L) leaves, 6% rosmarinic acid, Salvia lavandulifolia and Salvia officinalis, L-Theanine, L-ergothioniene.
